(19) United States
(12) Patent Application Publication
(10) Pub. No.: US 2007/0191790 A1
(43) Pub. Date: Aug. 16, 2007

(54) Wire guide having distal coupling tip

(75) Inventors: Scott E. Eells, Bloomington, IN (US); Robert M. Eells, Bloomington, IN (US)

Correspondence Address: BRINKS HOFER GILSON & LIONE/CHICAGO/COOK, PO BOX 10395, CHICAGO, IL, 60610, US

(73) Assignee: Cook Incorporated, Bloomington, IN (US)

(21) Appl. No.: 11/706,548

(22) Filed: Feb. 15, 2007

Related U.S. Application Data
(60) Provisional application No. 60/773,944, filed on Feb. 16, 2006.

(51) Int. Cl. A61M 25/00; A61M 5/00

(52) U.S. Cl. 604/264

(57) ABSTRACT

A coupling wire guide structured to be slidably coupled to a previously introduced wire guide. The coupling wire guide includes a main body and a tip portion. The tip portion includes an insert member and a distal tip. The insert member is connected to the main body, and the distal tip is connected to the insert member. The insert member defines an insert passageway having a laterally opening proximal port. The distal tip defines a tip passageway having a distal port, the tip passageway being in communication with the insert passageway. The proximal port, insert passageway, tip passageway and distal port are sized to receive the previously introduced wire guide.

### FIELD OF THE INVENTION

This invention relates generally to a wire guide for use in intracorporeal procedures, and more particularly relates to the construction of a wire guide to be coupled to a previously introduced wire guide for assistance during interventional procedures in vessels with proximal tortuosity, or as a more substantial wire guide for angioplasty procedures, stenting procedures, and other device placement procedures and their related devices.

### BACKGROUND OF THE INVENTION

Proximal tortuosity of the vasculature is problematic for all medical catheter devices such as atherectomy devices, angioplasty devices, stent delivery devices, and filter delivery devices. Wire guides are therefore typically used to navigate the vasculature of a patient during percutaneous interventional procedures. Once the wire guide has been introduced, it may then be used to introduce one or more medical catheter devices. Thus, most wire guides are typically 0.014 inches in diameter and have a lubricious coating to enhance wire guide introduction movement. Conventional 0.014 inch floppy wire guides must have sufficient flexibility and torque control for navigation through tortuous vessels. At the same time, the wire guide must have a certain amount of rigidity to pass through lesions, straighten extremely tortuous vessels, and provide support to medical catheter devices that are introduced over the wire guide.

Accordingly, wire guides are subjected to potentially conflicting requirements. Conventional 0.014 inch floppy wire guides are usually sufficient for navigation of moderately tortuous vessels. However, in some situations the wire guide tip may prolapse away from the catheter device which it is supporting. For example, balloon angioplasty in vessels with proximal tortuosity has been associated with a higher incidence of acute complications and procedural failure due to the inability to cross lesions with a conventional floppy wire guide, and due to the inability of the wire guide to provide adequate support to the balloon catheter. Heavy-duty wire guides, on the other hand, are generally not well suited as primary wire guides because of their stiffness and potential for causing injury to the vessel during introduction.

It may therefore be desirable to use conventional floppy wire guides for navigation of tortuous vessels, and then supplement the conventional wire guide with a supplemental stiffer wire guide. The supplemental wire guide will straighten out the vessel curves and ease further wire guide movement. Additionally, the supplemental wire guide provides greater support and enhances the tracking of balloons, stents, stent delivery devices, atherectomy devices, and other medical catheter devices as compared to a conventional floppy wire guide. This technique is commonly referred to as the “Buddy Wire” technique, details of which are disclosed in U.S. patent application Ser. No. 11/081,146, filed Mar. 16, 2005.

However, the feeding of the supplemental wire guide parallel to the first wire guide is an exacting and time consuming process in which additional difficulties are encountered. For example, the second wire guide can cork screw or coil around the first wire guide, which may result in immobilization or unintended movement of the first wire guide, which in turn may require the retraction and re-feeding of the supplemental wire guide and/or the primary wire guide. Moreover, if retraction of the supplemental wire guide is necessary, either of the wire guides may become contaminated and the entire process may need to be restarted with sterile components. The time consumed by this process can be critical to the success of the procedure. Additionally, when traversing through the heart of a patient, and particularly the ostium, the larger open space of the heart makes identical placement of the supplemental wire guide somewhat difficult.

Accordingly, there exists a need to provide a supporting wire guide for intracorporeal procedures that may be easily and reliably traversed through the vasculature to a position proximate a previously introduced wire guide.

### BRIEF SUMMARY OF THE INVENTION

The present invention provides a wire guide for intracorporeal procedures that may be easily and reliably traversed through the vasculature to a position proximate a previously introduced wire guide. The wire guide is a coupling wire guide that is structured to be slidably coupled to the previously introduced wire guide. In one embodiment constructed in accordance with the teachings of the present invention, the coupling wire guide generally includes a main body and a tip portion. The tip portion includes an insert member and a distal tip. The insert member is connected to the main body, and the distal tip is connected to the insert member. The insert member defines an insert passageway having a laterally opening proximal port. The distal tip defines a tip passageway having a distal port, the tip passageway being in communication with the insert passageway. The proximal port, insert passageway, tip passageway and distal port are sized to receive the previously introduced wire guide.

According to more detailed aspects, the insert member preferably has an outer diameter less than or about equal to an outer diameter of the main body. In this manner, the coupling wire guide is provided with a profile similar to standard (non-coupling) wire guides, while still providing a coupling function to achieve secure and reliable translation of the coupling wire guide along the previously introduced wire guide. Likewise, the distal tip has an outer diameter about equal to the outer diameter of the insert member. Most preferably, the main body, insert member and distal tip are co-axial. It will also be recognized that the main body and distal tip may also comprise a solid or tubular mandrel, multiple wound designs or any other wire guide construction.

In another embodiment constructed in accordance with the teachings of the present invention, a method is provided for forming the coupling wire guide. The method includes the steps of providing a wire guide having a wound outer wire, stretching a distal tip portion of the wire guide to create an intermediate space, and positioning an insert member within the intermediate space. Thus, according to this method, the main body and distal tip of the coupling wire guide are formed by a coiled outer wire. According to more detailed aspects of this embodiment, the stretching step generally includes increasing the pitch in the area of the intermediate space. Preferably, the positioning step includes injection molding the insert member in the intermediate space and over the outer wire of the wire guide. The method may also include the step of forming an insert passageway in the insert member. Finally, the method may further comprise the step of forming a distal port in the distal end cap of the wire guide.

### BRIEF DESCRIPTION OF THE DRAWINGS

The accompanying drawings incorporated in and forming a part of the specification illustrate several aspects of the present invention, and together with the description serve to explain the principles of the invention. In the drawings:

FIG. 1 is a cross-sectional view of a coupling wire guide constructed in accordance with the teachings of the present invention;

FIG. 2 is a cross-sectional view of another coupling wire guide constructed in accordance with the teachings of the present invention; and

FIG. 3 is a cross-sectional view of yet another coupling wire guide constructed in accordance with the teachings of the present invention.

### DETAILED DESCRIPTION OF THE INVENTION

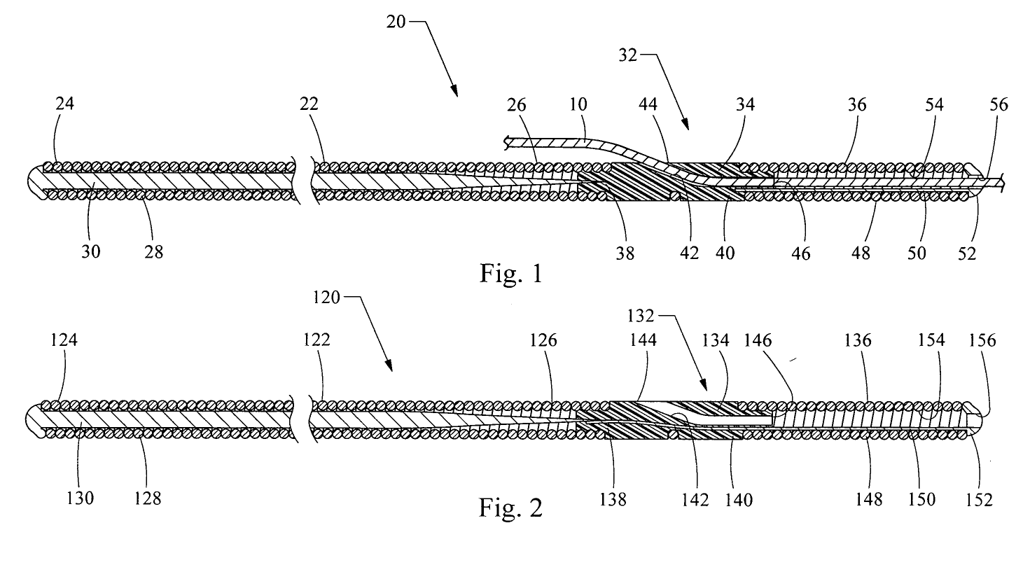

Turning now to the figures, FIG. 1 depicts a cross-sectional view of a coupling wire guide 20 constructed in accordance with the teachings of the present invention. While wire guides are often used in percutaneous interventional procedures, it will be recognized by those skilled in the art that the wire guide of the present invention may also be employed non-percutaneously, such as in endoscopic or other intracorporeal procedures. The coupling wire guide 20 generally includes a main body 22 having a proximal end 24 and a distal end 26. The main body 22 is constructed by an outer wire 28 which is disposed over an inner mandrel 30, preferably by winding or coiling the outer wire 28 over mandrel 30, as is well known in the art. However, it will be recognized by those skilled in the art that the main body 22 may take many forms as many types of wire guides are known in the art, including solid wire, tubular wire, weaved or interlaced wires, coiled wires and combinations thereof, such as is shown in U.S. Pat. No. 5,243,996, the disclosure of which is hereby incorporated by reference in its entirety. Preferably, the outer wire 28 is constructed of a metal such as stainless steel or nitinol (Ni—Ti alloy), although any biocompatible material may be used.

The coupling wire guide 20 also includes a tip portion 32 at the distal end 26 of the main body 22. The tip portion generally includes an insert member 34 linked to a distal tip 36. The insert member 34 is preferably formed of a biocompatible plastic, and most preferably a thermoplastic which may be injection molded such as polytetrafluorethylene (PTFE), polyethylene ether ketone (PEEK), polyamide, polyimide, polyurethane, polyethylene and nylon, including multi-layer or single layer constructions with or without reinforcement wires, coils or filaments. The distal tip 36 is constructed of a coiled wire, and preferably the same outer wire 28 of the main body 22, as will be discussed in more detail below.

The insert member 34 generally includes a proximal end 38 connected to the distal end 26 of the main body 22, and a distal end 40 connected to the distal tip 36. It can be seen in FIG. 1 that the proximal end 38 is connected to both the outer wire 28 and the tapered end of mandrel 30. Similarly, the distal end 40 is connected to the outer wire 28 as well as a safety wire 50 forming a portion of the distal tip 36. The distal tip 36 generally includes a distal end cap 52 connected to the outer wire 28, as well as connected to the safety wire 50. The main body 22, insert member 34, and distal tip 36 are co-axially aligned. By way of this construction, torqueability of the wire guide 20 and its distal tip 36 is maintained.

The insert member 34 includes an insert passageway 42 which defines a proximal port 44. The proximal portion of the insert passageway 42 is angled relative to an axis of the main body 22. The laterally facing proximal port 44 and angled portion of the insert passageway 42 ease the transition of a previously introduced wire guide 10 away from the coupling wire guide 20. The distal tip 36 defines a tip passageway 54 having a distal port 56. Specifically, the outer wire 28 defines the tip passageway, while an aperture in the end cap 52 defines the distal port 56. The tip passageway 54 is in communication with the insert passageway 42 for allowing the previously introduced wire guide to pass therethrough. That is, the distal portion of insert passageway 42 opens axially to the tip passageway 54. Thus, the proximal port 44, insert passageway 42, tip passageway 54, and distal port 56 are sized to receive the previously introduced wire guide 10, which typically has a diameter of about 0.014 or 0.018 inches. Therefore, it can also be seen that the wire guide 20 generally has an outer diameter of about 0.035 to about 0.038 in order to provide appropriately sized passageways 42, 54 and ports 44, 56.

It will be recognized by those skilled in the art that the insert member 34 has an outer diameter less than or about equal to an outer diameter of the main body 22. Likewise, the distal tip 36 has an outer diameter about equal to the outer diameter of the insert member 34. By the term “about equal”, it is generally meant that the diameters are within 10% of each other. In this manner, the wire guide 20 is provided with a coupling tip portion 32 which does not have an increased diameter, thereby providing a normal profile consistent with non-coupling wire guides. At the same time, coupling function is provided in a manner for secure and reliable interconnection of the coupling wire guide 20 to a previously introduced wire guide (not shown). Furthermore, by spacing the proximal port 44 and the insert 34 from the distal tip 36 of the guide 20, a flexible distal tip 36 is provided to assist in the navigation of the wire guide 20, whether coupled or de-coupled from a previously introduced wire guide 10.

Turning to FIG. 2, an alternate embodiment of a coupling wire guide 120 is depicted in accordance with the teachings of the present invention. Similar to the prior embodiment, the wire guide 120 includes a main body 122 having proximal and distal ends 124, 126, and defined by an outer wire 128 disposed over an inner mandrel 130. The insert member 134 includes a proximal end 138 connected to the main body 122, a distal end 140 connected to a distal tip 136, and defining an internal passageway 142 having a proximal port 144. Similarly, the distal tip 136 includes an outer wire 128, a safety wire 150 and an end cap 152 defining the distal port 156.

Unlike the prior embodiment, here the safety wire 150 extends through a majority of the insert member 134 and is connected to the narrowed tip of the mandrel 130. Preferably, the safety wire 150 and mandrel 130 are connected by soldering, although welding, adhesives or other well known bonding techniques may be employed. In this manner, improved torque control over the distal tip 136 is provided while securely connecting the main body 122, insert member 134 and distal tip 136.

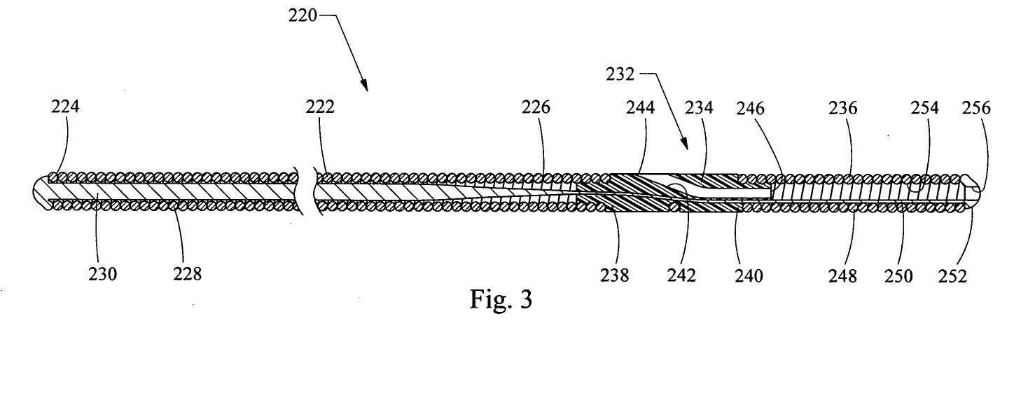

FIG. 3 depicts yet another embodiment of a coupling wire guide 220 constructed in accordance with the teachings of the present invention. Similar to the prior embodiments, the wire guide 220 includes a main body 222 having proximal and distal ends 224, 226, and defined by an outer wire 228 disposed over an inner mandrel 230. The insert member 234 includes a proximal end 238 connected to the main body 222, a distal end 240 connected to a distal tip 236, and defining an internal passageway 242 having a proximal port 244. Similarly, the distal tip 236 includes an outer wire 228, a safety wire 250 and an end cap 252 defining the distal port 256.

In this embodiment, the mandrel 230 narrows as it approaches the insert member 234, and then itself forms the safety wire 250. That is, the mandrel 230 and safety wire 250 are unitarily formed as a single piece. The safety wire 250 may be formed by centerless grinding the end of the mandrel 230. As in the prior embodiment, this inter-connection of the mandrel 230 and safety wire 250 provides a secure link between the main body 222, insert member 234 and tip portion 236, while also improving torque control over the tip portion 236 for improved navigation of the coupling wire guide 220.

The present invention also provides a method for forming a coupling wire guide in accordance with the teachings of the present invention. The method includes the first step of providing a wire guide having a wound outer wire. Then, a distal tip portion of the wire guide is stretched to create an intermediate space. Stated another way, a distal tip portion of the wire guide is manipulated to increase the pitch between adjacent coils in the intermediate space. This intermediate space is generally the space filled by the insert member 34 depicted in FIG. 1. With the intermediate space formed, the method includes positioning an insert member, such as member 34, within the intermediate space. Preferably, the insert member is injection molded, and preferably over molded over the outer wire 28 of the wire guide. As shown in the figures, the outer wire 28 continues to traverse through the insert member 34. Through the molding process, the insert member can be shaped to define an insert passageway 42 having an appropriate size for passage of a previously introduced wire guide. However, the insert passageway 42 could also be formed after the formation of the insert member 34 through any well known technique for removing material. The method also includes the formation of the distal port 56 in the end cap 52 using similar techniques. Those skilled in the art will recognize that this method of the present invention permits the adaptation of existing wire guides to coupling wire guides, as well as providing a secure inter-connection between the main body 22, insert member 34 and distal tip 36. Nonetheless, the coupling wire guide can be formed in many other ways, including through use of separate main body 22 and distal tip 36 pieces, which as previously discussed may take many forms.

Accordingly, those skilled in the art will recognize that the coupling wire guide of the present invention is quickly and easily coupled to a previously introduced wire guide and reliably traversed through the vasculature. At the same time, the coupling wire guide is provided with a generally constant profile that is consistent with non-coupling wire guides, thereby improving their use when not coupled to another wire guide. Likewise, the distal tip of the wire guide is securely connected to the main body, maintaining suitable torque control for the navigation of tortuous pathways.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. A coupling wire guide for coupling to a previously introduced wire guide during intracorporeal procedures, the coupling wire guide comprising:
a main body;
a tip portion including an insert member and a distal tip, the insert member connected to the main body, the distal tip connected to the insert member;
the insert member defining an insert passageway having laterally opening proximal port;
the distal tip defining a tip passageway having a distal port, the tip passageway in communication with the insert passageway; and
the proximal port, insert passageway, tip passageway, and distal port sized to receive the previously introduced wire guide.

2. The coupling wire guide of claim 1, wherein the insert member has an outer diameter less than or about equal to an outer diameter of the main body.

3. The coupling wire guide of claim 2, wherein the distal tip has an outer diameter about equal to the outer diameter of the insert member.

4. The coupling wire guide of claim 1, wherein the main body, insert member and distal tip are co-axial.

5. The coupling wire guide of claim 1, wherein the main body is formed by a coiled outer wire, and wherein the distal tip is also formed by the coiled outer wire, and wherein the insert member is positioned in an area of increased pitch of the coiled outer wire.

6. The coupling wire guide of claim 1, the main body defining a guide axis, wherein the insert passageway is angled relative to the guide axis.

7. The coupling wire guide of claim 1, wherein the main body comprises an outer wire disposed over a mandrel.

8. The coupling wire guide of claim 7, wherein the insert member is connected to both the outer wire and the mandrel.

9. The coupling wire guide of claim 7, wherein the distal tip includes a safety wire, and wherein the safety wire is connected to the mandrel.

10. The coupling wire guide of claim 9, wherein the safety wire is unitarily formed with the mandrel and extends through the insert member.

11. The coupling wire guide of claim 1, wherein the distal tip includes a safety wire connected to at least one of the insert member and the main body.

12. The coupling wire guide of claim 1, wherein the distal tip includes an end cap defining the distal port.

13. The coupling wire guide of claim 12, wherein the distal tip includes a safety wire connected to the end cap.

14. A method for forming a coupling wire guide structured for coupling to a previously introduced wire guide, the method comprising:
providing a wire guide having a wound outer wire;
stretching a distal tip portion of the wire guide to create an intermediate space; and
positioning an insert member within the intermediate space.

15. The method of claim 14, further comprising the step of forming an insert passageway in the insert member, the insert passageway having laterally opening proximal port.

16. The method of claim 15, wherein the positioning step includes forming the insert member with an outer diameter less than or about equal to an outer diameter of the wire guide.

17. The method of claim 14, wherein the positioning step includes co-axially aligning the insert member with the wire guide.

18. The method of claim 14, wherein the positioning step includes injection molding the insert member in the intermediate space and over the outer wire of the wire guide.

19. The method of claim 14, wherein the wire guide includes a distal end cap, and further comprising the step of forming distal port in the distal end cap.

20. The method of claim 14, wherein the stretching step includes increasing the pitch in the area of the intermediate space.
